(19) United States
(12) Plant Patent Application Publication
(10) Pub. No.: US 2025/0048945 P1
(43) Pub. Date: Feb. 6, 2025

(54) Pistachio Hybrid Rootstock Named '19PaPi-SY'

(50) ;

(71) Applicant: Sierra Gold Nurseries, Inc., Yuba City, CA (US)

(72) Inventors: Micah Stevens, Yuba City, CA (US); Reid Robinson, Yuba City, CA (US); Charles Brannon, Yuba City, CA (US)

(73) Assignee: Sierra Gold Nurseries, Inc., Yuba City, CA (US)

(21) Appl. No.: 18/230,583

(22) Filed: Aug. 4, 2023

(51) Int. Cl. A01H 5/10; A01H 6/00

(52) U.S. Cl. CPC A01H 5/10; A01H 6/00
USPC PLT/152

(57) ABSTRACT

A new and distinct pistachio tree named ‘19PaPi-SY’ developed for use as a rootstock and notable for its vigor, amenability to in vitro propagation, asphyxia tolerance and compatibility with Pistacia vera scions.

Latin name: Pistacia atlantica x Pistacia integerrima.

Variety denomination: ‘19PaPi-SY’

### BACKGROUND OF THE VARIETY

‘19PaPi-SY’ is a new and distinct pistachio tree developed for use as a rootstock. ‘19PaPi-SY’ was selected from among seedlings obtained from a controlled cross of female parent Pistacia atlantica (not patented) and male parent Pistacia integerrima (not patented) carried out at Yuba City, California. The ‘19PaPi-SY’ pistachio tree was observed to be more vigorous than standard pistachio rootstock ‘UCB-1’ (not patented) and was selected for propagation and further observation in 2020. The first asexual propagation of the variety was carried out by in vitro propagation in 2020 at Yuba City, California. The variety has since been asexually reproduced through successive generations and has been observed to remain true to type.

### BRIEF DESCRIPTION OF THE VARIETY

‘19PaPi-SY’ was first selected for its vigor as compared to ‘UCB-1’ pistachio rootstock. A molecular genetic marker in ‘19PaPi-SY’ predicts broad environmental adaptability of increased vigor. ‘19PaPi-SY’ has been found to be more amenable to in vitro propagation than ‘UCB-1’, with an in vitro multiplication rate of about 133% of the ‘UCB-1’ multiplication rate. Asphyxia tolerance in ‘19PaPi-SY’ exceeds that of ‘UCB-1’ and ‘Platinum’ (not patented) commercial pistachio rootstocks. ‘19PaPi-SY’ is further useful as a rootstock due to its scion compatibility with Pistacia vera edible kernel pistachio varieties.

### BRIEF DESCRIPTION OF THE PHOTOGRAPHS

FIG. 1 shows a tree of the claimed variety;

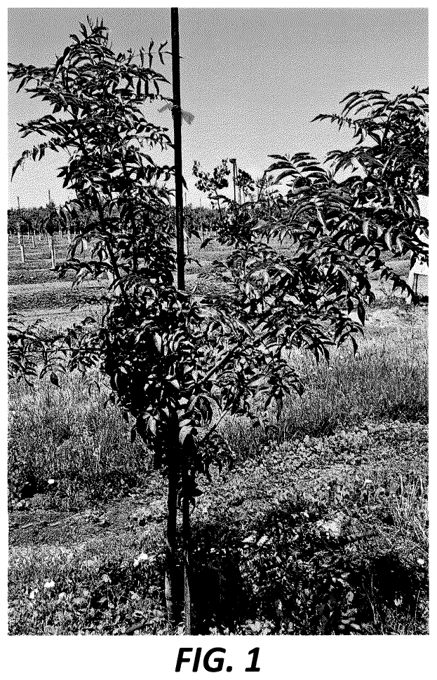

FIG. 2 shows a branch and leaves of the claimed variety;

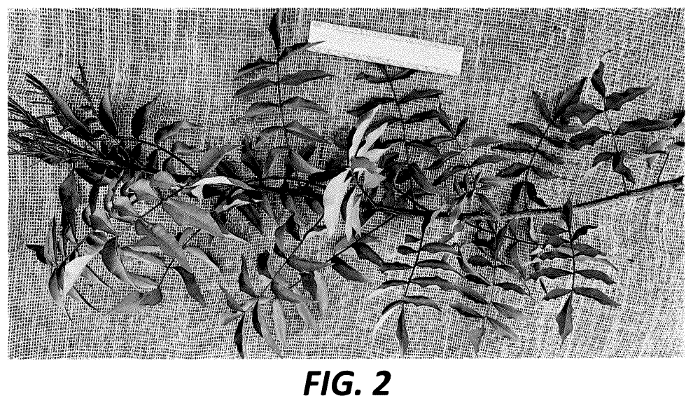

FIG. 3 shows the upper surface of leaves of the claimed variety; and

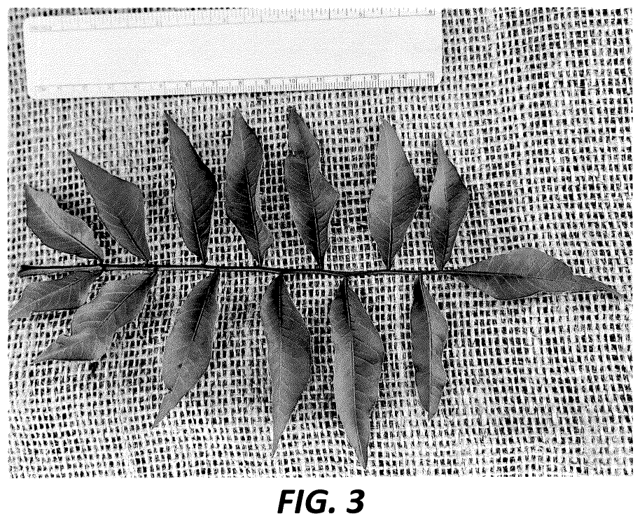

FIG. 4 shows the lower surface of leaves of the claimed variety.

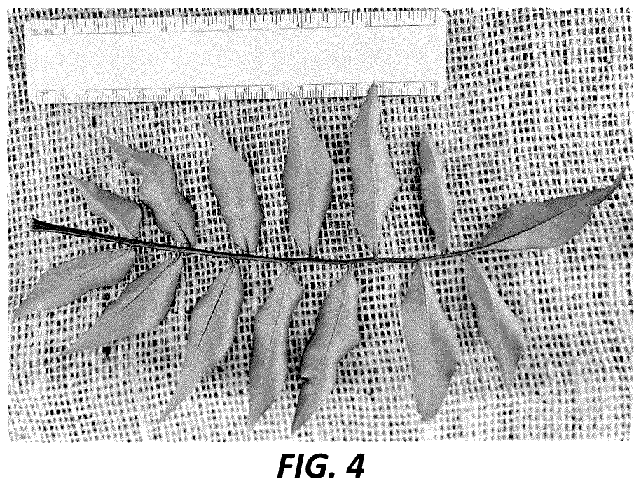

### DETAILED BOTANICAL DESCRIPTION OF THE VARIETY

The following-detailed botanical description is based on observations made at Yuba City, California during the 2023 growing season of 3-year-old trees grown on their own roots. All colors are described according to The R.H.S. Colour Chart (Royal Horticultural Society, London, 6^{th} edition). It should be understood that the characteristics described will vary somewhat depending upon cultural practices and climatic conditions, and will vary with location and season. Quantified measurements are expressed as an average of measurements taken from a number of individual plants of the new variety. The measurements of any individual plant or any group of plants of the new variety may vary from the stated average.
Tree:
   Height.—1.8 m.
   Trunk diameter at 20 cm above the soil.—24.6 mm.
   Bark texture.—Slightly rough.
   Bark color.—Grey-brown 199C.
   Lenticel length.—1.9 cm to 2.0 mm.
   Lenticel width.—<1 mm.
   Lenticel shape.—Flat.
   Lenticel color.—Greyed-orange 165C.
   Lenticel density per cm².—0 to 6.
One year old shoot:
   Length.—70 cm to 79 cm.
   Diameter.—8.0 mm to 9.1 mm.
   Pubescence.—Absent.
   Color.—Greyed-red 178A.
   Lenticel length.—<1 mm.
   Lenticel width.—<1 mm.
   Lenticel shape.—Round.
   Lenticel color.—Greyed-orange 165B.
   Lenticel density per cm².—0 to 8.
Leaves:
   Shape.—Alternate, even-pinnately compound.
   Length.—18.1 cm to 25.6 cm.
   Width.—12.1 cm to 15.6 cm.
   Length/width ratio.—1.2:1.
   Leaflet length.—7.1 cm to 8.2 cm.
   Leaflet width.—1.8 cm to 2.6 cm.
   Blade margin.—Entire.
   Apex shape.—Acute.
   Base shape.—Attenuate.
   Color of upper surface.—Green NN137B.
   Color of lower surface.—Yellow-green 146B.
   Attitude in relation to shoot.—Outward.
Petiole:
   Length.—3.8 cm.
   Diameter.—3 mm to 4 mm.
   Color.—Greyed-red 178A.
   Disease resistance/susceptibility.—Tolerant of Verticillium wilt.
   Pest resistance/susceptibility.—Testing is ongoing.

1. A new and distinct pistachio tree named ‘19PaPi-SY’ substantially as illustrated and described herein.
